(19) United States
(12) Patent Application Publication
(10) Pub. No.: US 2014/0235540 A1
(43) Pub. Date: Aug. 21, 2014

(54) ANDROGEN AND GONADOTROPIN TREATMENT IN FEMALES

(71) Applicants: Norbert Gleicher, New York, NY (US); David H. Barad, Closter, NJ (US); Dwyn V. Harben, Bryn Mawr, PA (US)

(72) Inventors: Norbert Gleicher, New York, NY (US); David H. Barad, Closter, NJ (US); Dwyn V. Harben, Bryn Mawr, PA (US)

(21) Appl. No.: 14/100,202

(22) Filed: Dec. 9, 2013

Related U.S. Application Data
(63) Continuation-in-part of application No. 13/043,266, filed on Mar. 8, 2011.
Application No. 13/043,266 is a continuation-in-part of application No. 12/575,426, filed on Oct. 7, 2009, now Pat. No. 8,501,718.
Application No. 12/575,426 is a continuation-in-part of application No. 12/610,215, filed on Oct. 30, 2009, now Pat. No. 8,501,719.
Application No. 12/610,215 is a continuation-in-part of application No. 12/123,877, filed on May 20, 2008, now Pat. No. 8,067,400.

(51) Int. Cl. A61K 38/24; A61K 31/5685

(52) U.S. Cl. CPC A61K 38/24; A61K 31/5685
USPC 514/9.9

(57) ABSTRACT

A method of treating a human female with an androgen and a gonadotropin to improve at least one of the human female's infertility, reproductive outcomes and oocyte yield is disclosed. The method may include treating the female with an androgen and a gonadotropin in combination. The androgen may be administered for more than six weeks and the gonadotropin may be administered in a regular low dosage over a period of time longer than two weeks. The method may include inducing ovulation in the female by administering gonadotropins to stimulate ovulation and/or induce ovulation. The method may include multiple additional inductions of ovulation, each within 120 days of the previous induction of ovulation.

This application is a continuation-in-part of U.S. patent application Ser. No. 13/043,266, filed on Mar. 8, 2011, which is a continuation-in-part of application Ser. No. 12/123,877, filed on May 20, 2008, now U.S. Pat. No. 8,067,400 B2, and Ser. No. 12/575,426, filed on Oct. 7, 2009, now U.S. Pat. No. 8,501,718 B2, and Ser. No. 12/610,215, filed on Oct. 30, 2009, now U.S. Pat. No. 8,501,719 B2, which are incorporated by reference herein in their entirety.

### BACKGROUND OF THE INVENTION

1. Field of the Invention

The present invention relates to the field of reproductive technology.

2. Description of the Related Art

The related art of assisted reproductive technology revolutionized the treatment of infertility. The most common assisted reproductive technology is in vitro fertilization (IVF), in which a woman's eggs are harvested and fertilized with a man's sperm in a laboratory. Embryos grown from the fertilized eggs are then chosen to be transferred into the female's uterus. In large part, assisted reproductive technology in females depends on ovarian stimulation and concurrent multiple oocyte development, induced by exogenous gonadotropins.

It is understood in the prior art that the follicles in the ovary, which develop into oocytes, are only sensitive to exogenous gonadotropins during the two week period immediately prior to ovulation. Accordingly, conventional ovarian stimulation is only performed in the prior art during the two week period immediately prior to ovulation.

To improve ovarian stimulation, infertile women often are treated with gonadotropin treatments, such as gonadotropin-releasing hormone (GnRH) flare protocols, that control gonadotropin secretion in the woman. Estrogen pre-treatment with concomitant growth hormone (GH) treatment sometimes is used to amplify intra-ovarian insulin-like growth factor-I (IGF-I) paracrine effect, which is expressed by granulosa cells and enhances gonadotropin action.

Exogenous Dehydroepiandrosterone (DHEA) improves ovarian stimulation in older women who respond poorly to gonadotropin treatments. DHEA is secreted by the adrenal cortex, central nervous system and the ovarian theca cells. DHEA is converted in peripheral tissue to more active forms of androgen or estrogen through biosynthesis. DHEA secretion during childhood is minimal but it increases at adrenarche and peaks around age 25, the age of maximum fertility, only to reach a nadir after age 60.

Females with diminished ovarian function (DOF) have decreased egg production and the eggs that are produced usually are of a poor quality. Further, females with diminished ovarian function often encounter difficulty becoming pregnant with or without IVF, experience long time periods to conception and/or have an increased possibility of miscarriage and/or an increased possibility of having high number/percentages of aneuploid embryos.

### BRIEF SUMMARY OF THE INVENTION

The present invention is directed generally to a method of treating a human female using an androgen and a gonadotropin to improve fertility and reproductive outcomes. One embodiment of the method includes administering an androgen in combination with a gonadotropin to a human female. The androgen may be DHEA, testosterone, DHEA sulfate (DHEA-S), androstenedione, any other androgen or a combination thereof. In all embodiments of the invention, the gonadotropin may be follicle stimulating hormone (FSH), luteinizing hormone, human menopausal gonadotropin, any other gonadotropin, or a combination thereof.

The present invention is further directed to a method of treating a human female to increase oocyte production. One embodiment of the method comprises administering an androgen and a gonadotropin daily or, at least, regularly to the female. The androgen is preferably administered for at least six weeks and the gonadotropin is preferably administered for a period longer than two weeks to improve the female's oocyte count. The periods of androgen administration and gonadotropin administration preferably overlap. The method further includes monitoring the female's follicle count. One embodiment includes stimulating ovulation after the female's follicle count has increased. In another embodiment, the gonadotropin is about 150 IU of follicle stimulating hormone per day. In another embodiment, the androgen is DHEA. In another embodiment, the androgen is testosterone and in other embodiments, other goanadotropins and/or androgens may be used. In all embodiments of the invention, inducing ovulation may include ovarian stimulation by administering a gonadotropin for a period of up to two weeks.

The present invention is further directed to a method of ovulation induction in a human female comprising administering an androgen to a female for at least six weeks, administering a gonadotropin during those six weeks to stimulate the female's ovaries, inducing ovulation and repeating the gonadotropin administration and ovulation induction less than 120 days after the first ovulation induction while continuing to administer the androgen between the first and second ovulation induction. In one embodiment, the androgen is DHEA. Furthermore, the gonadotropin administration and ovulation induction may be repeated less than 120 days after the second ovulation induction and again less than 120 days after each subsequent ovulation induction while continuing to administer the androgen between each ovulation induction. The androgen may be administered orally.

The length of time the androgen is administered to the female is at least six weeks. The androgen treatment may preferably continue for more than four months. The length of time the gonadotropin is administered to the female is a period longer than two weeks. The length of time the gonadotropin is administered to the female is preferably more than two months. The androgen treatment and the gonadotropin administration may continue for periods of time longer than four months or indefinitely until a desired condition is achieved.

### BRIEF DESCRIPTION OF THE DRAWINGS

FIG. 1 is a table showing improved ovulation induction with DHEA.

FIG. 2 is a graph showing an increase in the number of fertilized oocytes resulting from oocytes harvested from women with DHEA treatment.

FIG. 3 is a graph showing an increase in the number of fertilized oocytes resulting from oocytes harvested from women with at least 4 weeks of DHEA treatment.

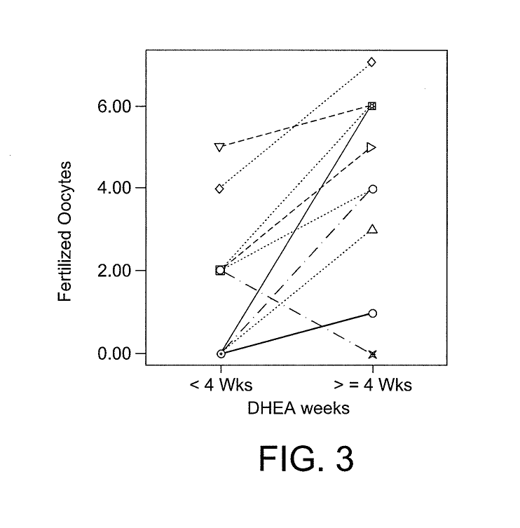

FIG. 4 is a chart showing chemical pathways of adrenal function.

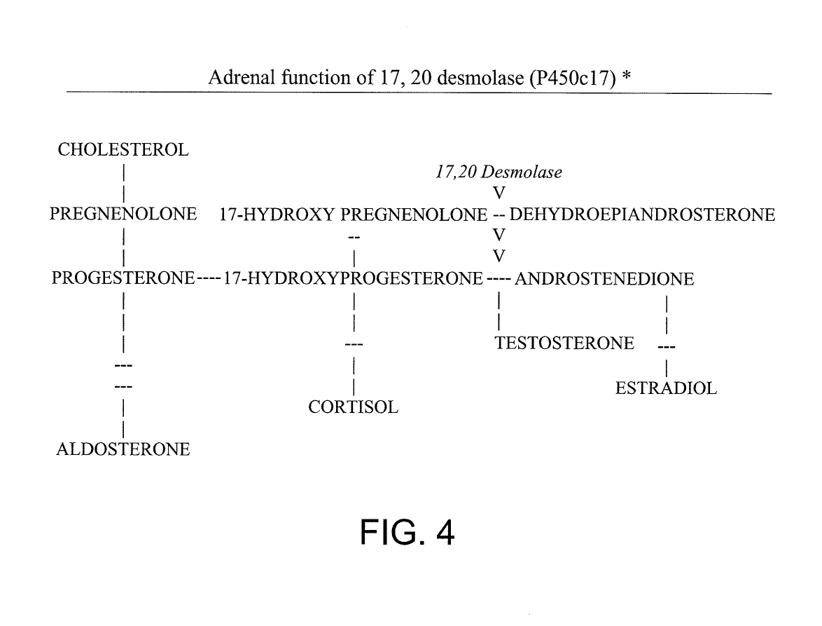

FIG. 5 is a graph comparing the number of oocytes per retrieval in patients with short intervals between ovarian stimulations and continuous gonadotropin exposure and patients with long intervals between ovarian stimulations and interrupted gonadotropin exposure.

FIG. 6 is a graph comparing the number of oocytes retrieved per cycle between females with differing ranges of total FSH dose per cycle.

FIG. 7 is a graph comparing the number of oocytes retrieved per cycle between females above and below an AMH serum level of 1.05 ng/mL.

### DETAILED DESCRIPTION OF THE INVENTION

The present invention is a method of increasing oocyte yield in human females by administering an androgen and a gonadotropin to the human female. One embodiment of the invention comprises administering androgen to the human female for a period of at least four months, or sixteen weeks, and performing multiple induced ovulation cycles in the human female. An induced ovulation cycle includes ovarian stimulation and ovulation induction. Each cycle is performed no later than 120 days after the previous cycle. Another embodiment of the invention comprises administering an androgen to the human female for a period of at least six weeks followed by administering an androgen and a gonadotropin for a period of longer than two weeks.

IVF is the most common assisted reproductive technology. When attempting IVF, older women produce few oocytes and yield few normal embryos, even when exposed to maximal gonadotropin stimulation. The decreased ability of older women to respond to ovulation-inducing medications shows that ovarian reserve declines with age. Even with multiple IVF cycles, older women produce few oocytes and yield few normal embryos, as compared to younger women, when exposed to maximal gonadotropin stimulation. This change in ovarian responsiveness is known as diminished ovarian reserve or diminished ovarian function.

In the present invention, to improve the number of eggs, the number of embryos, spontaneous pregnancy rates, IVF pregnancy rates, cumulative pregnancy rates and/or time to conception, a therapeutically effective amount of an androgen is administered in combination with a low dosage of a gonadotropin to a human female. Preferably, the human female is a premenopausal human female who may exhibit a diminished ovarian reserve. The androgen may be administered to a human female at a dose of between about 50 mg/day and about 100 mg/day, preferably between about 60 mg/day and about 80 mg/day, and more preferably about 75 mg/day. Further, the androgen may be administered in a time-release formulation, over the course of the day, or in a single dose. For example, the about 75 mg/day could be administered in a single dose of 75 mg or could be administered as 25 mg three times throughout the day. The follicle stimulating hormone may be administered to the human female at a dose of between approximately 75 IU per day and approximately 150 IU per day.

The androgen has a statistically significant effect on the number of eggs, the number of embryos, spontaneous pregnancy rates, IVF pregnancy rates, cumulative pregnancy rates and/or time to conception, after about six weeks of use, but its effect may continue to increase to about four months, about 16 weeks. The administration of the androgen may continue for a period of time longer than six weeks, preferably about four consecutive months, about 16 consecutive weeks, and further may continue past four consecutive months of use.

As used herein, administration of the androgen for a period of time, from at least one month to about six weeks to about four months, or 16 weeks, or longer, does not require a dose each and every day over this time span, although a daily dose is preferred. Rather, it is intended that the androgen be administered on a regular basis throughout the period of time, with daily administration being the most effective. A regular basis as used herein is any schedule of administration that provides sustained exposure to the administered compound and/or any schedule of administration that administers the administered compound on a substantially regular schedule. The dosage and regularity of androgen administration may be determined by one skilled in the art and may be adjusted based on its desired effects, the appearance of side effects or on other criteria. The androgen is preferably administered orally, although the androgen may be administered or delivered via other methods, such as, but not limited to, intravenously, intramuscularly and/or topically.

The effects of DHEA increase over time, and may reach peaks after approximately four to five months of supplementation. It is suggested that peaks may occur at four to five months because this time period is similar to the time period of a complete follicular recruitment cycle. Further, the effect of DHEA is suggested to reduce chromosomal abnormalities and thus substantially decreasing miscarriage rates in human females.

Treatment with an Androgen Improves Ovulation

Treatments with androgens, alone or in conjunction with other hormones, increase a woman's response to ovulation induction, measured in both oocyte and embryo yield. Androgen treatment may be an adjunct to ovulation induction. DHEA taken orally for at least about one month, preferably for about four months, before optionally initiating gonadotropin treatment, is beneficial for preparing the ovaries for gonadotropin stimulation. An increased response to ovulation induction may be obtained by combining gonadotropins and DHEA in treatment for at least about a six week period and ideally a four month, or sixteen week, period before an IVF cycle.

Young ovaries are characterized by large numbers of antral follicles (secondary or later stage follicles showing an antrum) and a low rate of atresia. In contrast, older ovaries have few antral follicles, high rates of atresia and exhibit increasing “resistance” to ovulation induction. Older women have decreased oocyte quantity and quality, produce fewer high quality embryos and have lower implantation and pregnancy rates. Most follicular atresia occurs after the primordial follicle resumes growth but before the follicle is gonadotropin responsive enough for recruitment. An induced delay in the onset of atresia can preserve follicles for future ovulation. Such a delay, or arrest, of the atretic process is known among anovulatory women with polycystic ovary syndrome (PCO). These women's follicles remain steroidogenicaly competent and show evidence of increased aromatase activity compared to like-sized follicles from normal ovaries. Aromatase transforms precursor hormones into active hormones. Follicular hypersecretion of DHEA, which is typical of PCO, is associated with increased aromatase activity.

Peter R. Casson and associates, at John E. Buster's research group (Baylor Medical College, Houston, Tex.) were the first to suggest therapeutic benefits from DHEA supplementation in women with diminished ovarian reserve (DOR). They first reported that micronized DHEA offers the potential of postmenopausal steroidal replacement, adjunctive to estrogen. In adrenal and ovarian steroidogenesis, DHEA is an intermediate product in the conversion of cholesterol to the sex hormones, testosterone and estradiol. They, however, demonstrated that in postmenopausal women this conversion is not symmetrical and favors androgens. While testosterone after DHEA supplementation increases, estradiol remains low. In further exploring androgen deficiency in menopause, Casson and associates demonstrated that DHEA has immunomodulatory effects, an observation now well recognized and therapeutically explored in treating autoimmune diseases.

Casson and associates later demonstrated that vaginally administered DHEA, while delivering equivalent hormone, substantially diminishes bioconversion in comparison to oral micronized product. Casson and associates also showed that abnormally low DHEA secretion is increased in effect by ovarian hyperstimulation. This work led to a study of a case series of women with poor response to ovarian stimulation with gonadotropins, in which Casson and associates reported improvements in ovarian response after DHEA supplementation. Their rationale for this study was previously observed increases in insulin-like growth factor 1 (IGF-1) after DHEA supplementation. They speculated that DHEA may improve oocyte yields via IGF-1, because IGF-1 increases following growth hormone administration had been suggested to improve oocyte yields via IGF-1.

Casson and associates, in Casson, et al., Dehydroepiandrosterone Supplementation Augments Ovarian Stimulation in Poor Responders: a Case Series, Human Reproduction, October 2000, at 2129, did not outright suggest a benefit of DHEA treatment in females with DOR. Instead, they claimed that DHEA supplementation may augment conventional ovarian stimulation with gonadotropins (i.e. gonadotropin stimulation for a period of less than two weeks) in poor responders and result in improved oocyte yields. This conclusion was reached in six IVF cycles based on investigation of only five proven poor responders, under the age of 41 years, and with baseline follicle stimulating hormone (FSH) under 20 mIU/ml. After receiving 80 mg of micronized DHEA for two months, all study subjects demonstrated improved responsiveness in comparison to a prior unsupplemented cycle, characterized by increased peak estradiol and improved peak estradiol/gonadotropin dosage ratios. In addition, one patient delivered a twin pregnancy.

The next published report on DHEA supplementation appeared five years later and described the experiences of one index patient. Like Casson et al, this index patient showed improvement in oocytes yields, that seemed far greater than initially reported by the Baylor group. (Barad D H and Gleicher N., 2005) Gleicher 2005 showed that increasing the length of DHEA supplementation continuously improved oocyte (and embryo) numbers and speculated about possible causes: DHEA over time could have cumulative benefits and/or could have synergistic effects with gonadotropin stimulation, which the index patient underwent almost monthly in pursuit of nine consecutive cycles. Cumulative effects over time suggest a DHEA effect on follicular recruitment cycles in their total length, while synergistic effects with gonadotropin stimulation appeared a possibility based on the prior reports that gonadotropins augment adrenocortical DHEA-S secretion. Gleicher 2005 did not show the mechanism of action of DHEA or conclusively show a synergistic effect with gonadotropin stimulation. Further studies, discussed below, show the mechanisms of action of androgen treatment on ovulation and ovarian environment in human females.

Mechanisms of Androgen Effects on Ovulation

Hodges et al. suggested that treatments can be developed to reduce the risk of age-related aneuploidy by influencing meiotic chromosome segregation. Hodges et al., Experimental Evidence That Changes in Oocyte Growth Influence Meiotic Chromosome Segregation, Human Reproduction, May 2002, at 1171. Hodges et al. believe that major disturbances in chromosome alignments on the meiotic spindle of oocytes (congression failure), responsible for aneuploidy, result from the complex interplay of signals regulating folliculogenesis. These failures in folliculogenesis regulation increase the risk of non-disjunction errors.

The conventional understanding in the art that oocytes age, which increases aneuploidy, is incorrect. Instead, the unrecruited egg is suspended in time and does not age to a significant degree. The cause of aneuploidy increase with age is not aging of oocytes but aging of the ovarian environment within which oocytes undergo folliculogenesis. By correcting age-related changes in the ovarian environment (declining androgen levels is only one amongst many such changes), aneuploidy levels can be maintained at levels closer to those in younger women. Reduction of miscarriage rates in androgen pregnancies to those of average, fertile patient populations supports this new understanding.

The effect of androgens on the entire cycle of folliculogenesis, the follicular maturation cycle, is suggested by the continuous improvement in androgen effects over at least five to six months of androgen treatment. The continuous improvement strongly supports an androgen effect that increases as developing follicles are exposed to androgen for longer and longer periods of time. Other modes of androgen action contemplated in the art include increases in ovarian IGF-1 to cause androgen effects. IGF-1 appears reduced in poor responders to ovarian stimulation.

Androgens also enhance ovarian function. Androgens have been demonstrated to increase follicular recruitment. Increasing intrafollicular androgen levels augment granulose cell AMH and inhibin-B production. Androgen receptors are present in ovarian stroma, granulose cells of primordial follicles, primary follicles and at more advanced stages of folliculogenesis. Ovarian androgens, but not estrogens, correlate with systemic inflammation during ovarian stimulation with gonadotropins.

Androgen levels throughout a female's ovulation cycle and thereafter can affect ovulation and pregnancy success. Frattarelli and associates initially reported that testosterone levels in human females on day three of their ovulation cycle at or under 20 ng/dL are associated with poorer IVF pregnancy rates. They later reported only an association with IVF stimulation parameters but no association with pregnancy success. Lossl et al. published contradictory papers, one claiming and one refuting that treatment with aromatase inhibitors (which increases intrafollicular androgens) improves embryo quality. Contradictory results were reported by French investigators on short-term transdermal testosterone administration, with Massin et al reporting no benefit, while Balasch's group in two publications found transdermal testosterone supplementation has beneficial effects on ovarian resistance to stimulation with gonadotropins.

Primordial oocytes that make up the unrecruited egg pool in a woman's true ovarian reserve, most likely do not really age. An incorrect belief that such primordial oocytes age is the present understanding in the art. Primordial oocytes, similar to cryopreserved gametes, hibernate at metabolic rates close to zero until recruited into folliculogenesis. Once recruited, they pass the various stages of folliculogenesis within the age-dependent environment of a woman's ovary, which changes significantly as women age.

This new understanding is that the unrecruited eggs do not age, rather the ovarian environment, within which oocytes mature through folliculogenesis, ages. Under this new understanding, androgen supplementation of an androgen-deficient ovarian environment causes environmental “rejuvenation,” and, for upcoming cohorts of follicles, improves folliculogenesis to levels usually found only in younger women.

This new understanding allows a new treatment of DOR, that maximizes ovarian conditions for the entire period of folliculogenesis, rather than only during the last two weeks of gonadotropin sensitivity. The new understanding and treatment allows the female's reproductive lifespan to be significantly extended. While egg numbers irreversibly decline with advancing age, even menopausal ovaries still contain follicles and oocytes. In the squirrel monkey, older animals, immediately prior to cessation of reproduction, still demonstrate an abundance of well-differentiated granulosa cells. Assuming that unrecruited oocytes maintain their youth and that an aged ovarian environment can be rejuvenated, smaller, but healthier, egg cohorts from a follicular development cycle may allow for pregnancy into very advanced female ages.

Aged eggs cannot be returned to youth. An aged environment, however, can be improved with appropriate pharmaceutical therapies. Androgen represents a first treatment which has this rejuvenating effect on the ovarian environment. By “correcting” the ovarian environment, androgen allows for a follicular maturation process in some patients that mimics that of younger women. As a consequence, egg and embryo quality is improved, pregnancy rates are higher and miscarriage rates are lower, as one would expect in younger women.

Androgen Treatment in Combination with Continuous Gonadotropin Treatment Improves Oocyte Yields

Females treated with androgen for diminished ovarian functional reserve (DFOR) and also continuously and concurrently treated with a gonadotropin, such as FSH, produce more oocytes per ovulation cycle than females treated with androgen but not continuously treated with gonadotropins. Stimulated ovulation in an IVF cycle includes gonadotropin treatment in the last ten days to two weeks prior to stimulated ovulation. Among women treated with androgen and undergoing multiple IVF cycles during such treatment, ovarian stimulation cycles spaced less than about 120 days apart result in improved oocyte yields in later ovarian stimulation cycles.

Closely spaced ovarian stimulation cycles provide long-term gonadotropin exposure. This long-term gonadotropin exposure in combination with androgen treatment resulted in improved oocyte yields in females undergoing closely spaced ovarian stimulation cycles as compared to females undergoing ovarian stimulation cycles at longer intervals. For the purposes of this disclosure, a closely spaced ovarian stimulation cycle means one stimulation cycle followed by another stimulation cycle less than 120 days later, which may in turn be followed by another stimulation cycle less than 120 days from the second stimulation cycle, and so on.

The conventional understanding in the art is that follicles generally become sensitive to gonadotropins during the last two weeks of folliculogenesis. Based on this understanding, the last two weeks of folliculogenesis are conventionally described as the “gonadotropin-sensitive” stage of folliculogenesis. Pharmacologic infertility treatments, practically since inception, concentrated on the last two weeks of folliculogenesis, the “gonadotropin-sensitive” stage. Based on this prior understanding of follicular sensitivity to gonadotropins, standard ovarian stimulation uses large doses of gonadotropin in a period of time shorter than two weeks and only at the end of folliculogenesis. Androgens affect growing follicles at earlier stages of development than stage that is generally understood to be the “gonadotropin-sensitive” stage of folliculogenesis.

Gonadotropins can also affect growing follicles at a stage before the follicles were previously understood to be sensitive to gonadotropins. Therefore, a novel technique to improve oocyte yields in ovulation involves administration of gonadotropins over a period of time that is significantly greater than the last two weeks of folliculogenesis. Such administration of gonadotropins may also be in lower dosages than used in conventional ovarian stimulation. Such administration of gonadotropin may occur daily or regularly at a longer or shorter interval than daily. The administration of gonadotropins may be followed by one or more induced ovulation cycles or may allow natural ovulation. The administration of gonadotropins may be for a period of longer than two weeks, over a month, about six weeks, over two months, about four months or sixteen weeks, or may be sustained until a desired end-state is achieved. Such desired end-state may be, without limitation, the collection of a certain number of oocytes, the creation of a certain number of embryos, the implantation of a certain number of embryos and/or the achievement of a successful pregnancy in the female.

The following examples are to be construed as merely illustrative and not limitative of the disclosure in any way.

### Example 1

### Improved Ovulation after DHEA Treatment

Example 1 shows the effects of androgen treatment in combination with conventional IVF treatment. These effects are significantly increased in the treatment of the present invention, as shown in Example 2.

In the example study, a 43 year old woman, Patient A, undergoing IVF with banking of multiple cryopreserved embryos for future aneuploidy screen and transfer, is administered an androgen, namely DHEA. In ten months, she undergoes eight treatment stimulation cycles while continuously improving her ovarian response, resulting in oocyte and embryo yields far beyond those previously seen in a woman her age. Patient A's history is unremarkable except for two previous malarial infections. She is allergic to sulfa medications and has a history of environmental allergies. Her surgical history includes umbilical hernia repair at age one and cholecystectomy at age 21. She had used oral contraceptives for over 10 years. She has no history of irregular menstrual cycles.

Day three serum FSH and estradiol (E2) in her first IVF cycle are 11 mIU/ml and 18 pg/ml, respectively. In subsequent cycles, her baseline FSH is as high as 15 mIU/ml. She is given an ovulation induction protocol which is prescribed for patients with evidence of decreased ovarian reserve. Briefly, the protocol includes the following medications: norethindrone acetate tablets (10 mg) for 10 days, starting on day two of menses, followed three days later by a “microdose” dosage of 40 pg of leuprolide acetate, twice daily, and, after another three days, by 600 IU of FSH (Gonal-F; Ares-Serono, Geneva, Switzerland) daily. Peak serum E2 concentration on day nine of stimulation is 330 pg/ml. Following injection of 10,000 IU human chorionic gonadotropin (hCG), she undergoes oocyte retrieval. Only one oocyte is obtained and one embryo is cryopreserved.

Because of the poor response to ovulation stimulation, she is advised to consider donor oocyte or embryo donation. She rejects both options. She starts a second cycle using the same stimulation protocol with one exception: instead of 600 IU of FSH daily, Patient A received 450 IU of FSH and 150 IU of human menopausal gonadotropin (HMG, Pergonal, Ares-Serono, Geneva, Switzerland). This stimulation protocol is continued in identical fashion for the remaining cycles. However, two weeks before starting her second cycle, she begins administration of 75 mg per day of oral micronized DHEA. The date on which she begins administration of 75 mg per day of oral micronized DHEA is Oct. 6, 2003.

Methods of Example 1

The eight treatment cycles are divided into three groups to allow statistical comparison: pre-initiation and very early use of DHEA (early-cycles 1 and 2), initial cycles (mid=cycles 3-5), and later cycles (late=cycles 6-8). Comparison between these categories is by one-way analysis of variance (ANOVA) and multiple comparisons by Student-Neuman-Keuls (SNK) test. The homogeneity of variances and orthogonal linear contrasts were tested to compare groups and polynomial contrast to test for linear and quadratic trends. All outcomes are presented as mean±1 standard deviation. Rate of change of oocyte counts, cryopreserved embryos and (log transformed) peak estradiol between subsequent cycles is estimated by linear regression.

Embryos are evaluated by the embryologists on day three post-insemination for cell-count and grading. Embryo grading is based on a 1 to 4 scale depending on symmetry, percent fragmentation and appearance of the cytoplasm. All viable embryos are cryopreserved.

Statistics are performed using SPSS for Windows, Standard version 10.0.7 (SPSS Co., Chicago, Ill.). Assay of E2 and FSH are performed using the ACS: 180 chemoluminescence system (Bayer Health Care LLC, Tarrytown. N.Y.).

Results of Example 1

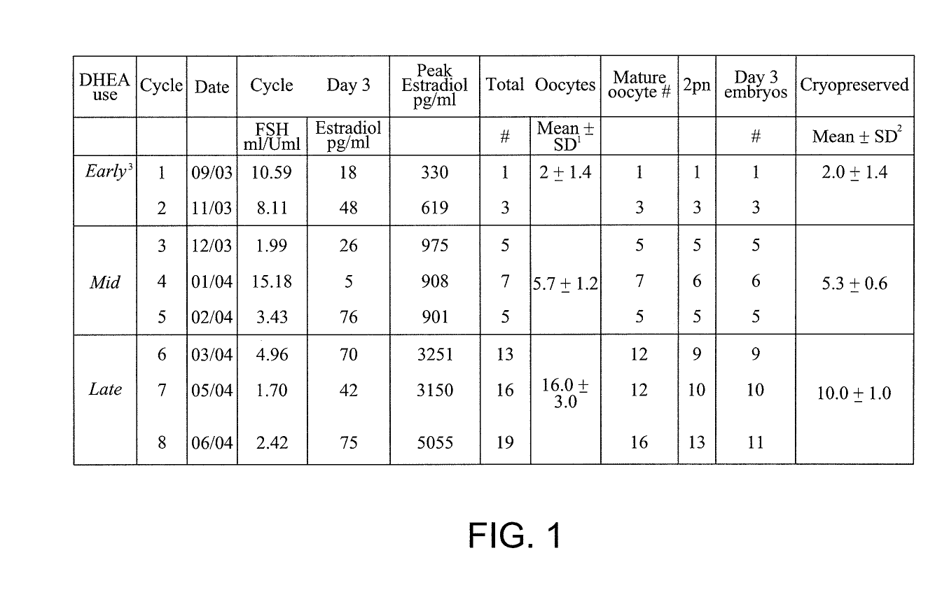

The results of ovulation induction with DHEA are displayed in FIG. 1. After eight stimulation cycles and approximately eight months of DHEA treatment, Patient A produced 19 oocytes and 11 cryopreservable embryos. A total of 50 viable embryos were cryopreserved.

Significantly more oocytes (p=0.001) and cryopreserved embryos (p<0.001) are obtained in the late cycles (cycles 6-8, 4+ consecutive months of DHEA treatment) compared to the combined early and mid cycles (cycles 1-5, 0-4 consecutive months of DHEA treatment). There is no significant difference in average embryo cell count (6.83±1.37 vs. 7.2±1.15) or morphology (3.6±0.5 vs. 3.7±0.5) between early and mid compared to late cycles. Peak E2, total oocyte, and embryos cryopreserved increase linearly from cycle to cycle, as shown in FIG. 1. Oocyte yield increase 2.5±0.34 oocytes per cycle (p<0.001), cryopreservable embryo yield increase 1.4±0.14 embryos per cycle (p<0.001) and (log) peak E2 increase 0.47±0.06 (p<0.001) across treatment cycles.

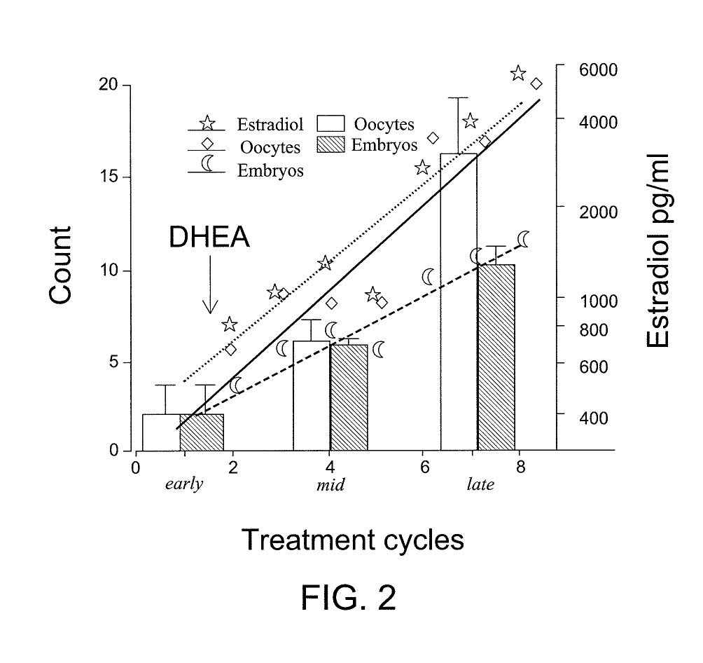

The linear increase in (log) peak E2 shown in FIG. 2 represents a cycle to cycle rate of increase from 123 pg/ml/cycle to 1491 pg/ml/cycle over the eight cycles of treatment. After adjusting for cycle day, the (harmonic) mean E2 is 267 pg/ml (95% confidence intervals (CI) 143 to 498 pg/ml) in the early phase, 941 pg/ml (95% CI 518 to 1712 pg/ml) in the mid phase, and 1780 pg/ml (95% CI 1121 to 2827 pg/ml) in the late phase of treatment. Each of these homogeneous subsets is significantly different from the other (p<0.05) by SNK multiple comparison testing.

The dramatic increase in oocyte and embryo yield experienced by this 43 year old woman is completely surprising and unexpected. Patient A's post-DHEA response to ovulation induction became more like that of a younger woman with PCO, than that of a 43 year old woman. With DHEA treatment, Patient A produced 49 embryos of high enough quality to undergo cryopreservation. Sixty percent of those embryos were produced in the last three cycles of treatment, which took place after at least about four consecutive months after starting treatment. After producing only one embryo prior to starting DHEA treatment, Patient A improved by an order of magnitude and produced 13 oocytes and 9 embryos in a cycle after at least about four consecutive months of DHEA treatment, 16 oocytes and 10 embryos in a cycle after at least about five and a half consecutive months of DHEA treatment, and 19 oocytes and 11 embryos in a cycle after at least about seven consecutive months of DHEA treatment. The increasing numbers of cryopreservable embryos following DHEA supplementation indicate improved embryo quality due to DHEA supplementation.

### Example 2

### Repeated Ovarian Stimulation Cycles in Conjunction with Androgen Treatment

Overview of Example 2

Androgens are understood to improve follicle maturation and ovarian reserve. It is reported that at least one function of androgens at early follicle stages is increasing the sensitivity of granulosa cells to follicle stimulating hormone (FSH). A study investigated the results of repeated ovarian stimulation cycles in women undergoing androgen treatment. The study found that repeated ovarian stimulation cycles spaced less than 120 days apart resulted in higher oocyte yields in later cycles than repeated ovarian stimulation cycles spaced 120 days apart or more than 120 days apart.

Details of Example 2

Supported by animal and human data, androgens are now recognized as beneficial to follicle maturation. (Gleicher, et al., The role of androgens in follicle maturation and ovulation induction: friend or foe of infertility treatment?, Reproductive Biology and Endocrinology, Aug. 17, 2011, at 116). Androgen receptors (ARs) appear on an increasing percentage of human follicles as the follicles move from the transitional stage to the primary and secondary follicle stages. Granulosa cell specific androgen receptor knockout (ARKO) mice provide evidence of reduced follicle progression and increased follicle atresia compared to wild type mice at these early stages of follicle maturation. ARs appear to play a critical role in normal mammalian follicle development.

At least one function of androgens at these early follicle stages is increasing the sensitivity of granulosa cells to follicle stimulating hormone (FSH). Therefore, it appears that androgens and FSH at these early follicle stages act synergistically.

At least six weeks of androgen supplementation is required to achieve significant benefits in women with DOR, though benefits are cumulative up to approximately four to five months. Best pregnancy results are obtained if androgen supplementation is combined with in vitro fertilization (IVF), and the results of androgen supplementation where the androgen is DHEA directly correlate with how well DHEA converts to testosterone.

Therefore, a study was designed to determine whether steady FSH exposure in association with androgen supplementation results in better oocyte yields than androgen exposure alone. DHEA was selected as a representative androgen. The same results are believed to be achieved when other androgens are used, such as dehydroepiandrosterone sulfate, testosterone and androstenedione, and therefore methods in accordance with the invention cover alternative androgens to DHEA.

140 women who underwent at least three IVF cycles, while continuously on DHEA supplementation, were studied. These patients were subdivided into groups of women who in parallel were “continuously” exposed to gonadotropins (Group 1, n=68) and not continuously exposed (Group 2, n=17). Group 1 was defined as continuously exposed to FSH because all three gonadotropin-induced IVF cycles took place within less than 120 days of each other. Group 2 patients were characterized as unexposed to continuous FSH since their intervals between cycles was equal to or exceeded 120 days. A 120 day interval was chosen as cut off because this is approximately the time for a small growing follicle to move from a primary to an antral stage follicle. 55 women were excluded from the study either because a pregnancy or transfer of cryopreserved embryos interrupted DHEA supplementation or excessively delayed continuous treatments.

As mentioned, the 120 day interval between IVF cycles is based on the time for a small growing follicle to move from a primary to an antral stage follicle. As this time varies, for example, for different women, an improvement to the method in accordance with the invention would be to determine the specific time, for each woman, that it takes a small growing follicle to move from a primary to an antral stage follicle. This determination may be made in advance of the IVF cycles by techniques known to those skilled in the art to which the invention pertains. As a preliminary stage then, the time it takes for a small growing follicle to move from a primary to an antral stage follicle for a specific patient is determined. Then the IVF cycles are based on this time. This time may be greater than or less than 120 days, but for the purposes of the disclosure herein, it will be assumed that an average, standard or regular time for follicle development is 120 days.

Once diagnosed with DFOR, patients receive a uniform supplementation protocol with 25 mg of pharmaceutical grade, micronized DHEA, TID, uninterrupted until pregnancy (second, normally rising human chorionic gonadotropin level) or termination of fertility treatment attempts with autologous oocytes. This uninterrupted administration is preferably in the form of an, at least, daily dose. Deviation from the daily administration may reduce the efficiency of the technique, but it is not unforeseen that a daily regimen may be difficult for every patient to follow.

The administration of 25 mg of DHEA is believed to be a preferred dosage. However, the dosage may vary from 25 mg and may be in a range from between about 25 mg/day and about 100 mg/day. The parameters of the range and specific dosage depend on factors known to those skilled in the art to which the invention pertains.

After at least six weeks of DHEA supplementation, a first IVF cycle is initiated with the patient's first menses. All DFOR patients receive identical ovarian stimulation in a microdose agonist protocol (leuprolide acetate, Lupron™, Abbot Pharmaceuticals, North Chicago, Ill., USA). Ovarian stimulation always involves a preponderance of about 300 to about 450 IU of FSH (products of various manufacturers, depending on patient preference and insurance circumstances) and about 150 IU of a human menopausal gonadotropin (hMG) product (mostly, Repronex™, Ferring Pharmaceuticals, Parsippany-Troy Hills, N.J., USA).

Ovarian stimulation comprises pharmaceutical compounds or protocols to stimulate oocyte production by the ovaries. Ovulation induction comprises pharmaceutical compounds or protocols to induce ovulation and includes ovarian stimulation. Example compounds and protocols are disclosed above and further compounds and protocols are known in the art.

The administration of 150 IU of gonadotropin is believed to be a preferred dosage. However, the dosage may vary from 150 IU and may be in a range from between about 75 IU and about 150 IU. The parameters of the range and specific dosage depend on factors known to those skilled in the art to which the invention pertains.

Results of Example 2

IVF cycle outcomes were assessed in every patient's first, second and third stimulation cycle, based on oocyte yields, cancellation rates and embryos transferred.

Statistical analysis was performed using SPSS version 21.0 (IBM SPSS, Chicago Ill.). Chi-square tests were used to compare proportions. Continuous variables were presented as means±standard deviations (SD), and tested either by Student's t-test and/or analysis of variance. All tests were two-tailed, and a P-value of P<0.05 was considered statistically significant.

Changes in oocyte production across cycles were evaluated using a general linear model for repeated measures. Mixed models with repeated measures were used to test the effect of age and total gonadotropin dosage as covariates, with a factor identifying sets of cycles, based on </≧120 day intervals between the cycles. Pregnancy rates, quite obviously, could not be compared between the two groups since patient selection in this study allowed for pregnancies to occur only in 3^{rd} cycles.

Patient characteristics and primary infertility diagnoses are listed in Table 1. The primary infertility diagnosis of all women was DFOR. As the table demonstrates, mean age, body mass index (BMI), FSH, and AMH of study and control groups were similar. Mean oocyte yields for all cycles were also similar between groups, 2.87±3.03 for the study group and 3.49±3.23 for the control group.

TABLE 1
Patient characteristics

| Cycle interval | <120 days | ≧120 days |
|---|---|---|
| N | 68 | 17 |
| Age (years) | 40.50 ± 4.45 | 41.59 ± 3.16 |
| BMI (kg/m2) | 23.73 ± 3.9 | 23.00 ± 4.07 |
| FSH (mIU/mL) | 19.6 ± 17.9 | 16.7 ± 21.4 |
| AMH (ng/mL) | 0.42 ± 0.41 | 0.54 ± 0.40 |
| Gonadotropin | 6720 ± 2403 | 6209 ± 2646 |
| dosage/IVF cycle | | |
| Oocyte yield/ | 2.87 ± 3.03 | 3.49 ± 3.23 |
| IVF cycle | | |
| Race* (n/%) | | |
| Caucasian | 49 (72%) | 10 (65%) |
| African | 10 (15%) | 3 (12%) |
| Asian | 9 (13%) | 4 (24%) |

Repeated measures ANOVA confirmed that in Group 1 age (p<0.001), FSH dose (P=0.05), embryos transferred (P=0.004) and oocytes retrieved (P=0.001) significantly increased across the three cycles, while percentages of cycles with no oocytes retrieved (P=0.024) and canceled cycles (P=0.006) decreased across the three cycles of treatment for group 1 and increased for group 2, as shown in Table 2.

TABLE 2
Oocyte yields in 1^{st} - 3^{rd} cycles among patients who completed three cycles
of treatment with < or ≧120 days interval between cycles.

| | | | | | P value |
|---|---|---|---|---|---|
| | | Cycles of treatment | | | (linear |
| | N | 1^{st} cycle | 2^{nd} Cycle | 3^{rd} Cycle | trend) |
| Time < 120 days | | | | | |
| Age (years) | 68 | 40.2 ± 4.4 | 40.4 ± 4.3 | 40.5 ± 4.3 | 0.001 |
| FSH Dose (units) | 68 | 6519 ± 2202 | 6486 ± 2344 | 7381 ± 2355 | 0.05 |
| Cycles with no | 68 | 23 (33.8)^{a} | 17 (25.0) | 11 (16.2)^{a} | 0.024 |
| Oocytes | | | | | |
| Oocyte (total) | 68 | 2.81 (2.02-3.60)^{b,c} | 3.84 (2.88-4.80)^{b} | 4.50 (3.48-5.53)^{c} | 0.001 |
| Embryos transferred | 68 | 1.10 (0.78-1.42)^{b} | 1.37 (0.99-1.74) | 1.59 (1.2-1.95)^{b} | 0.004 |
| Cancelled Cycles | 68 | 32.4% (23-42)^{d} | 18% (8-27) | 13% (4-23)^{d} | 0.006 |
| Time ≧ 120 days | | | | | |
| Age (years) | 17 | 41.6 ± 3.2 | 42.2 ± 3.0 | 42.5 ± 3.2 | 0.001 |
| FSH Dose (units) | 17 | 6965 ± 2712 | 6520 ± 2288 | 5070 ± 2714 | 0.08 |
| Cycles with no | 17 | 2 (11.8) | 4 (23.5) | 5 (29.4) | 0.22 |
| Oocytes | | | | | |
| Oocyte (total) | 17 | 4.35 (2.78-5.92) | 3.35 (1.78-4.93) | 2.77 (1.19-4.33) | 0.185 |
| Embryos transferred | 17 | 1.24 (0.62-1.85) | 1.06 (0.44-1.67) | 0.82 (0.21-1.44) | 0.60 |
| Cancelled Cycles | 17 | 0 | 0 | 18% (7-29) | 0.028 |

Values are presented as means (95% confidence intervals) or ±standard deviation;
^{a}P = 0.03;
^{b}P = 0.003;
^{c}P = 0.001;
^{d}P = 0.018.

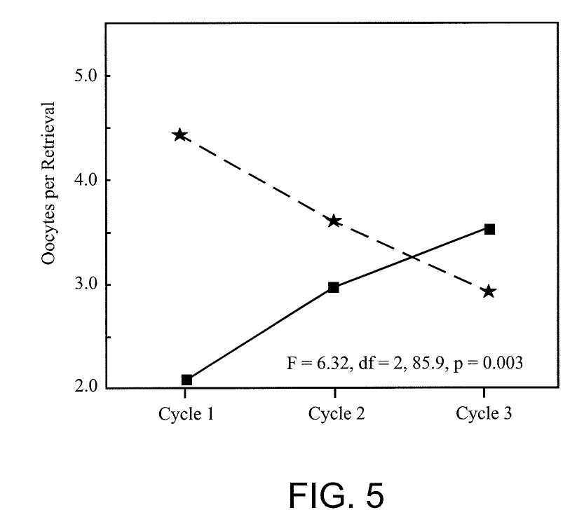

Although FSH dose also increased across the three cycles, there was no significant interaction between FSH dosage and cycles of treatment on number of oocytes retrieved. A linear mixed model with repeated measures was used to adjust for the effects of age and FSH dosage administered during ovarian stimulation across the three cycles of treatments F=6.32, df=2, 85.9, P=0.003 (FIG. 5). In this model a highly significant interaction was seen in Group 1 between repeated cycles of treatment and cycle interval of <120 days. Women with cycle interval of >120 days (Group 2) did not experience an increase in oocytes from cycle to cycle. They, indeed, experienced a nominally almost significant decrease in oocytes across the three cycles (FIG. 5).

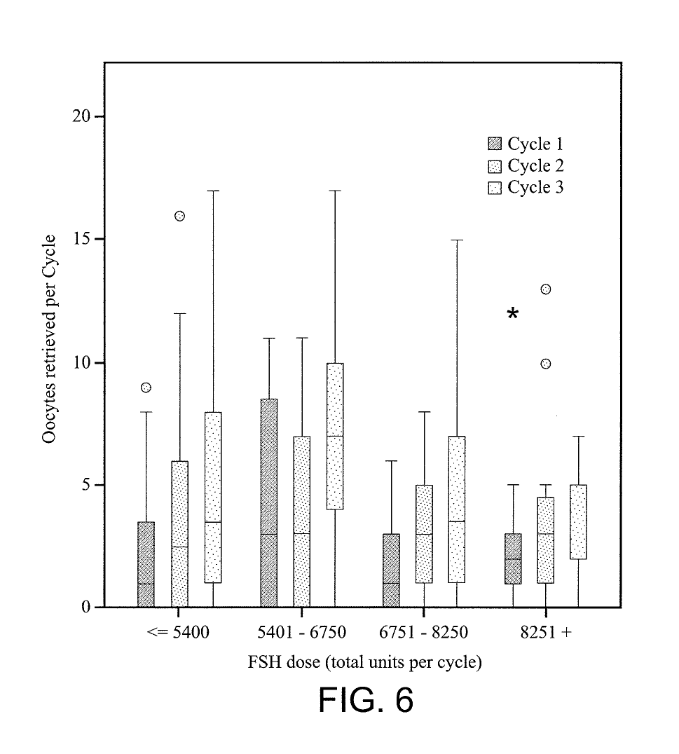

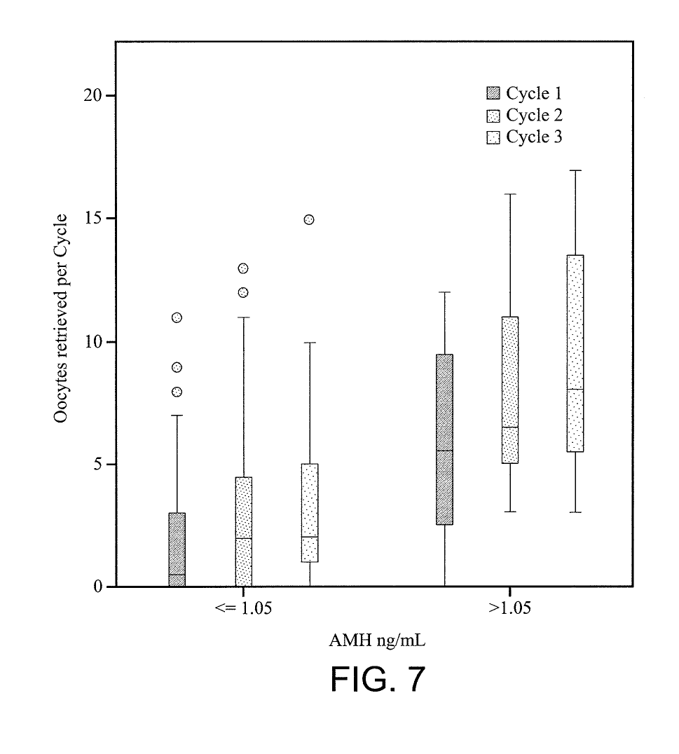

Oocyte yields in Group 1 increased from cycle to cycle independently of gonadotropin dosage administered for ovulation induction. (FIG. 6). Sub-dividing Group 1 patients further into poor and good prognosis patients, based on an AMH cut off of 1.05 ng/mL, the increase in oocyte yields was similar in both sub-groups. (FIG. 7).

The definition of both study groups did not allow for assessment of pregnancies in first and second IVF cycles since, by patient selection definitions, they had to be zero. Considering such an adverse selection of patients, 4 clinical pregnancies in Group 1 patients (5.9%) in third cycles, therefore, represents a quite remarkable observation. In contrast, Group 2 patients experienced no pregnancies in third cycles.

Discussion of Example 2

This study shows a functional clinical synergism between androgens and gonadotropins/FSH in women with DFOR. Women who in addition to routine DHEA supplementation also were continuously exposed to FSH and hMG (Group 1) produced increasing oocyte yields from cycle one through cycle three, while women with DFOR, who were not continuously exposed (Group 2) did not, and actually demonstrated declines in oocyte numbers (FIG. 5).

This study suggests that synergism between FSH and androgens should also result in better IVF pregnancy chances. Such a conclusion is supported by the known association between oocyte numbers and pregnancy rate but also by the quite remarkable observation of 4 pregnancies in Group 1 and none in Group 2 in third IVF cycles. These numbers suggest a remarkable trend towards better pregnancy rates in patients synergistically exposed to FSH and androgens.

Keeping DHEA supplementation stable, variations in FSH supplementation between different patient groups may allow for a better understanding of FSH/androgen synergism at early follicle maturation stages in humans. Above noted studies support the potential diagnostic importance of long-removed hormone levels in assessing physiologic conditions of ovaries in early growing follicle stages, immediately after follicle recruitment. Example 2 supports the conclusion that early hormone levels permit inferences about FOR and IVF outcomes from early-stage follicle cohorts, even if IVF outcomes are weeks to months removed.

In order to stratify subjects for concomitant FSH exposure, subjects were separated into those with steady FSH exposure (Group 1) and those with interrupted FSH exposure (Group 2).

A cut off of 120 days between IVF cycles was chosen because this time period approximately represents small growing follicle stages, the time it takes for primary follicles to reach preantral stages. Small growing follicle stages are the stage in which FSH/androgen synergism in animal models was demonstrated.

FIG. 5 demonstrates constant FSH exposure in parallel to DHEA supplementation significantly increases oocyte yields from cycle to cycle, while absence of steady FSH exposure results in practically opposite effects. Moreover, FIG. 6 and FIG. 7 demonstrate positive effects from androgen/FSH synergism are similar at all severity levels of DFOR, whether defined by gonadotropin stimulation dosage (FIG. 6) or AMH levels (FIG. 7).

These results also further enhance understanding of androgen effects on ovaries in women with DFOR. Though they only refer to oocyte yields, better oocyte yields, as noted before, usually reflect better FOR, which in turn is suggestive of better pregnancy chances. Clinically, these data suggest that patients with DFOR may benefit from closely scheduled consecutive IVF cycles, taking advantage of androgen/goandotropin synergism. Such an approach is contradictory to current practice patterns, which favors breaks between cycles.

This study also supports a radically new ovarian stimulation protocol. This new protocol, in contrast to traditional practice over the last 50-60 years, is specifically targeted at the early stages of follicle maturation. While traditional ovarian stimulation protocols apply gonadotropin stimulation only to the approximately last two weeks, the so-called gonadotropin-sensitive stage of follicle maturation, this new protocol benefits from gonadotropin (FSH)/androgen synergism in the small growing follicle stages.

Because follicles at these stages require in excess of 120 days to reach readiness for natural or induced ovulation, such a protocol entails low dose FSH administration in the form of standard gonadotropin preparations, possibly combining FSH and hMG for its LH effect, at ranges of 75-150 IU daily. This gonadotropin administration would be combined with a standard dosage of androgen for two to three months to reach appropriate testosterone levels, advancing so-stimulated follicle cohorts into gonadotropin-sensitive stages of follicle maturation, at which point a routine stimulation could be initiated.

The patients studied in Example 2 were affected by objectively determined DFOR, based on rigid age-specific ovarian reserve (OR) criteria. Highly abnormal FSH levels of 19.6±17.9 and 16.7±21.4 mIU/mL and AMH levels of 0.42±41 ng/mL and 0.54±0.40 (Table 1), respectively, speak for themselves, and clearly indicate how adversely patients in this study were selected.

Synergistic DHEA/FSH supplementation positively affects FOR (i.e., small growing follicles), almost independently of severity of DFOR and, possibly, age. This is supported by low miscarriage rates at advanced ages following DHEA supplementation, even with the most severe forms of DFOR. Since an AMH value of 1.05 ng/mL discriminates at all ages between better and poorer IVF pregnancy chances in women with DFOR, the initially adversely selected patients were subdivided into women with more severe DOR (AMH≦1.05 ng/mL) and those with milder DOR and better IVF prognosis (AMH>1.05 ng/mL). As FIG. 7 demonstrates, they performed quite similarly following DHEA supplementation and IVF cycles.

Oocyte yields under androgen/FSH supplementation continue to increase through three consecutive IVF cycles. This suggests that women with severe DFOR who, despite limited pregnancy chances, wish to pursue IVF with autologous oocytes should consider at least three consecutive cycles. Whether the observed rise in oocyte yield continues with additional time of supplementation exposure to androgen/FSH remains to be determined.

Based on the foregoing studies, a method for improving oocyte yields in a human female in accordance with the invention includes administration of an androgen at least six weeks prior to a first ovulation induction. The date for the first ovulation induction may be set in any manner known to those skilled in the art, and is usually dependent on the female' menses. Ovulation induction includes ovarian stimulation by administering a gonadotropin for a period of two weeks or less ending on the date set for ovulation induction. The administration of the androgen is on a regular basis during this at least six week period, e.g., on a daily basis.

This at least six week period may be as long as four months or even as long as five months. The specific time of the period in which androgen is administered may be based on factors that relate to the female's reproductive characteristics and/or based on analysis of the effect of androgen on follicle maturation. It is possible to periodically or continuously determine the FSH level in the female and then adjust the administration of the androgen accordingly. It is possible to periodically or continuously determine the level of compounds that are affected by or derived from the androgen in the female, e.g., testosterone, and then adjust the administration of the androgen accordingly.

During the six week period, ovarian stimulation is performed. The ovarian stimulation is performed by administering a gonadotropin for a period of two weeks or less. The specific period of time in which the gonadotropin is administered may be based on factors that relate to the female's reproductive characteristics and is understood in the art to be two weeks or less and end on the set date for the first induced ovulation. The ovarian stimulation is considered in the art to be part of the induced ovulation process.

After the at least six week period is concluded and at the set date for the first induced ovulation arrives, ovulation is induced in accordance with any manner for initiating an induced ovulation. The administration of an androgen continues during the induced ovulation and thereafter on a regular basis, e.g., daily.

After a time period of less than 120 days after the first induced ovulation is initiated, a second induced ovulation is performed in accordance with any manner for initiating an induced ovulation. The second induced ovulation includes ovarian stimulation as understood in the art. The administration of an androgen continues during this second induced ovulation and thereafter on a regular basis, e.g., daily. This second induced ovulation initiation may be necessary if the female does not conceive during time period of 120 days after the first induced ovulation is initiated. By timing the period between the initiations of successive induced ovulations to be less than 120 days, the female is continuously treated with gonadotropin and the numbers of oocytes produced increases. Otherwise, and obviously, the second induced ovulation is not performed.

After a time period of less than 120 days after the second induced ovulation is initiated, a third induced ovulation is performed in accordance with any manner for initiating an induced ovulation. The third induced ovulation includes ovarian stimulation as understood in the art. The administration of an androgen continues during this third induced ovulation and thereafter on a regular basis, e.g., daily. This third induced ovulation initiation may be necessary if the female does not conceive during time period of 120 days after the second induced ovulation is initiated. By timing the period between the initiations of successive induced ovulations to be less than 120 days, the female is continuously treated with gonadotropin and the numbers of oocytes produced increases. Otherwise, and obviously, the third induced ovulation is not performed.

As a preliminary step to optimize the method, it is beneficial to determine that the female has diminished functional ovarian reserve. This determination may be based on diagnosis of the quantity of age-specific follicle stimulating hormone (FSH) and/or anti Mullerian hormone (AMH). The manner in which such a diagnosis is performed is known to those skilled in the art to which this invention pertains.

Although the description above generally relates to an in vivo fertilization, it is expected that the fertilization may alternatively be achieved in vitro. In this case, after the induced ovulation, the typical procedures for oocyte harvesting are undertaken. The oocyte may then be fertilized in a lab and then returned to the female's womb for development. Alternatively, the oocyte may be stored unfertilized or fertilized and then stored. Such storage may be by any means for oocyte or embryo storage. The advantageous concurrent administration of androgen and gonadotropin are thus achieved regardless of the whether the fertilization is in vivo or in vitro.

A second method for improving oocyte yields in a human female to in accordance with the invention includes initiating administration of an androgen and continuing said administration for at least six weeks. The administration of the androgen is on a regular basis during this at least six week period, e.g., on a daily basis. During this administration, a gonadotropin such as follicle stimulating hormone (FSH) is also administered for a period longer than two weeks. The administration of the gonadotropin is on a regular basis, e.g., daily. At a minimum, the gonadotropin should be administered at a frequency and in an amount sufficient to provide the female with steady exposure to gonadotropin.

The preferred period for the administration of the androgen is between two months and three months, may be as long as four months or may be continued until a certain outcome is obtained. The longer than two week period of gonadotropin administration is preferably at least two months, may be as long as four months or may be continued until a certain outcome is obtained. The certain outcome may be the harvesting of a certain number of oocytes, the creation of a certain number of embryos, clinical pregnancy or any other outcome. The specific time of the period in which androgen and the gonadotropin are administered may be based on factors that relate to the female's reproductive characteristics and/or based on analysis of the effect of androgen on follicle maturation. It is possible to periodically or continuously determine the FSH level in the female and then adjust the administration of the androgen and gonadotropin accordingly. It is possible to periodically or continuously determine the level of compounds that are affected by or derived from the androgen in the female, e.g., testosterone, and then adjust the administration of the androgen accordingly.

The longer than two week period during which androgen and FSH are concurrently administered may be considered a preparatory stage for subsequent steps. Such subsequent steps may include conventional ovarian stimulation and induced ovulation, oocyte harvesting following natural or induced ovulation, in vivo or in vitro fertilization following natural or induced ovulation or any other subsequent step benefiting from increased oocyte numbers.

While the foregoing written description of the invention enables one of ordinary skill to make and use what is considered presently to be the best mode thereof, those of ordinary skill will understand and appreciate the existence of variations, combinations, and equivalents of the specific exemplary embodiments thereof. The invention is therefore to be limited not by the exemplary embodiments herein, but by all embodiments within the scope and spirit of the appended claims.

I claim:

1. A method of treating a human female with an androgen and a gonadotropin to improve at least one of the human female's infertility, reproductive outcomes and oocyte yield, comprising the steps of:
administering an androgen to the human female on a regular basis for a period of at least six weeks; and
administering a gonadotropin to said human female on a regular basis for a period longer than two weeks wherein the androgen administration and gonadotropin administration overlap for at least two weeks.

2. The method of claim 1, wherein the androgen is selected from the group consisting of dehydroepiandrosterone, dehydroepiandrosterone sulfate, testosterone and androstenedione.

3. The method of claim 1, wherein from approximately 50 mg to approximately 100 mg per day of the androgen is administered to the female.

4. The method of claim 1, wherein the androgen administration is selected from the group consisting of topically, orally, intravenously and intramuscularly.

5. The method of claim 1, wherein the androgen is administered to the human female for at least eight weeks.

6. The method of claim 1, wherein the androgen is administered to the human female for at least four months.

7. The method of claim 1, wherein the androgen is administered substantially daily during said at least six weeks, and the gonadotropin is administered substantially daily during said period longer than two weeks.

8. The method of claim 1, wherein the androgen and the gonadotropin are administered to the human female on a regular basis until the human female is pregnant.

9. The method of claim 1, wherein the androgen and the gonadotropin are administered to the human female until a desired number of oocytes are harvested from the human female.

10. The method of claim 5, wherein the period of gonadotropin administration is at least eight weeks.

11. The method of claim 6, wherein the period of gonadotropin administration is at least eight weeks.

12. The method of claim 6, wherein the period of gonadotropin administration is at least three months.

13. The method of claim 6, wherein the period of gonadotropin administration is at least four months.

14. The method of claim 1, wherein the gonadotropin administration is selected from the group consisting of topically, orally, intravenously and intramuscularly.

15. The method of claim 1, further comprising monitoring the human female's oocyte count.

16. The method of claim 15, further comprising stimulating ovulation in the human female when said monitoring indicates that the human female's oocyte count has increased.

17. The method of claim 16, wherein the androgen and the gonadotropin are administered to the human female until a desired number of oocytes are harvested from the human female.

18. The method of claim 17, wherein the androgen and the gonadotropin are administered to the human female until a desired number of embryos created from oocytes harvested from the human female are implanted in the human female.

19. The method of claim 1, wherein the gonadotropin is selected from the group consisting of follicle stimulating hormone, luteinizing hormone, human menopausal gonadotropin and a combination of two or more of follicle stimulating hormone, luteinizing hormone and human menopausal gonadotropin.

20. The method of claim 1, wherein from approximately 75 IU to approximately 150 IU per day of the gonadotropin is administered to the female.

21. A method of ovarian stimulation, comprising the steps of:
administering an androgen to the human female on a regular basis for a period of between two and three months; and
administering a gonadotropin to the human female on a regular basis for more than two weeks during said period of between two and three months.

22. The method of claim 21, wherein the gonadotropin comprises follicle stimulating hormone and human menopausal gonadotropin.

23. The method of claim 21, wherein said androgen is selected from the group consisting of dehydroepiandrosterone, dehydroepiandrosterone sulfate, testosterone and androstenedione.

24. The method of claim 21, wherein from approximately 50 mg to approximately 100 mg of the androgen are administered daily to the human female on a regular basis.

25. The method of claim 21, wherein from approximately 75 IU to approximately 150 IU of the gonadotropin are administered daily to the human female on a regular basis.

26. The method of claim 21, wherein the androgen administration is selected from the group consisting of topically, orally, intravenously and intramuscularly.

27. The method of claim 21, wherein the gonadotropin administration is selected from the group consisting of topically, orally, intravenously and intramuscularly.

28. A method of improving oocyte yields in a human female, comprising:
administering an androgen on a regular basis to a human female for at least six weeks;
then, thereafter performing a first ovulation induction by administering at least one ovarian-stimulating compound and at least one ovulation-inducing compound;
performing a second ovulation induction by administering at least one ovarian-stimulating compound and at least one ovulation-inducing compound within 120 days of performing said first ovulation induction;
performing a third ovulation induction by administering at least one ovarian-stimulating compound and at least one ovulation-inducing compound within 120 days of administering said second ovulation induction; and
administering an androgen to the human female on a regular basis between said first ovulation induction and said third ovulation induction.

29. The method of claim 28, wherein the androgen is selected from a group consisting of dehydroepiandrosterone, dehydroepiandrosterone sulfate, testosterone and androstenedione.

30. The method of claim 28, wherein the ovarian-stimulating compound is selected from a group consisting of follicle stimulating hormone, luteinizing hormone and human menopausal gonadotropin.

31. The method of claim 28, wherein from approximately 50 mg to approximately 100 mg per day of the androgen are administered daily to the human female on a regular basis.

32. The method of claim 28, wherein the regular basis on which the androgen is administered is daily.

33. A method for improving conception in a human female, comprising:
administering an androgen on a regular basis to the female, and
simultaneously administering to the female a gonadotropin for substantially the same time period as the androgen is administered.

34. The method of claim 33, wherein the fertilization occurs in vivo, further comprising,
during a first ovulation induction, administering at least one ovulation-inducing compound to the female; and
administering an androgen on a regular basis to the female at the time of the first ovulation induction and on a regular basis for at least two months thereafter.

35. The method of claim 34, wherein the ovulation-inducing compound comprises a combination of gonadotropins.

36. The method of claim 34, further comprising:
during a second ovulation induction, administering at least one ovulation-inducing compound to the female less than 120 days after said first ovulation induction; and
administering an androgen at the time of the second ovulation induction and on a regular basis for at least two months thereafter.

37. The method of claim 36, wherein the ovulation-inducing compound comprises a combination of gonadotropins.

38. The method of claim 36, further comprising:
during a third ovulation induction, administering at least one ovulation-inducing compound less than 120 days after said second ovulation induction; and
administering an androgen at the time of the third ovulation induction and on a regular basis for at least two months thereafter.

39. The method of claim 38, wherein the ovulation-inducing compound comprises a combination of gonadotropins.

40. The method of claim 34, further comprising determining the time period during which to administer androgen and gonadotropin to the human female based on an expected date of the first ovulation induction, the determined time period being at least six weeks.

41. The method of claim 40, wherein the time period is about four months.

42. The method of claim 33, further comprising determining the time period during which to administer androgen and gonadotropin based on at least one parameter relating to follicle maturation in the human female.

43. The method of claim 34, wherein the fertilization occurs in vitro.

44. The method of claim 43, wherein the ovulation-inducing compound comprises a combination of gonadotropins.

45. The method of claim 43, further comprising:
during a second ovulation induction, administering at least one ovulation-inducing compound to the female less than 120 days after said first ovulation induction; and
administering an androgen at the time of the second ovulation induction and on a regular basis for at least two months thereafter.

46. The method of claim 45, wherein the ovulation-inducing compound comprises a combination of gonadotropins.

47. The method of claim 45, further comprising:
during a third ovulation induction, administering at least one ovulation-inducing compound less than 120 days after said second ovulation induction; and
administering an androgen at the time of the third ovulation induction and on a regular basis for at least two months thereafter.

48. The method of claim 47, wherein the ovulation-inducing compound comprises a combination of gonadotropins.

49. The method of claim 43, further comprising determining the time period during which to administer androgen and gonadotropin to the human female based on an expected date of the first ovulation induction, the determined time period being at least six weeks.

50. The method of claim 49, wherein the time period is about four months.

51. A method of improving all stages of folliculogenesis in a human female, comprising the steps of:
administering an androgen to the human female on a regular basis for a period of at least six weeks; and
administering a gonadotropin to said human female on a regular basis for a period longer than two weeks wherein the gonadotropin administration overlaps with said androgen administration for at least two weeks.

52. The method of claim 51, wherein the androgen is selected from the group consisting of dehydroepiandrosterone, dehydroepiandrosterone sulfate, testosterone and androstenedione.

53. The method of claim 51, wherein the gonadotropin is selected from the group consisting of follicle stimulating hormone, luteinizing hormone, human menopausal gonadotropin and a combination of two or more of follicle stimulating hormone, luteinizing hormone and human menopausal gonadotropin.

54. The method of claim 51 wherein from approximately 50 mg o approximately 100 mg per day of the androgen is administered to the female.

55. The method of claim 51, wherein from approximately 75 IU to approximately 150 IU per day of the gonadotropin is administered to the female.
